(19) United States
(12) Patent Application Publication
(10) Pub. No.: US 2023/0104993 A1
(43) Pub. Date: Apr. 6, 2023

(54) INTEGRATED 3D BIOPRINTING METHOD AND APPLICATION OF HARD MATERIALS AND CELLS FOR PREPARING BONE-REPAIR FUNCTIONAL MODULES AND BONE ORGANOIDS

(71) Applicant: CHONGQING MEDICAL UNIVERSITY, Chongqing (CN)

(72) Inventors: Xiaolin TU, Chongqing (CN); Yufei MA, Chongqing (CN); Jun LI, Chongqing (CN); Xian LI, Chongqing (CN); Zhurong TANG, Chongqing (CN); Zhengsong XIE, Chongqing (CN); Jie CHEN, Chongqing (CN); Xiaofang WANG, Chongqing (CN); Guangliang LIU, Chongqing (CN); Pengtao WANG, Chongqing (CN); Bo WANG, Chongqing (CN); Yisheng LUO, Chongqing (CN); Yangxi LIU, Chongqing (CN); Weimin GONG, Chongqing (CN); Molin LI, Chongqing (CN)

(73) Assignee: CHONGQING MEDICAL UNIVERSITY, Chongqing (CN)

(21) Appl. No.: 17/956,860

(22) Filed: Sep. 30, 2022

(30) Foreign Application Priority Data
Sep. 30, 2021 (CN) 202111169004.9

(51) Int. Cl. A61L 27/38; C12N 5/077; C12N 5/071; C12M 3/00; A61L 27/56; A61L 27/46; B33Y 10/00; B33Y 80/00; B33Y 70/00

(52) U.S. Cl. CPC A61L 27/3821; A61L 27/46; A61L 27/56; B33Y 10/00; B33Y 70/00; B33Y 80/00; C12M 21/08; C12N 5/0654; C12N 5/0697; A61L 2430/02; A61L 2430/40; C12N 2501/42; C12N 2513/00; C12N 2533/30; C12N 2533/54

(57) ABSTRACT

A technology of 3D printing integration of hard materials and cells, a preparation of bone-repair functional module with osteogenic microenvironment, bone organoid method and the application of quick repair of bone defects are provided. A preparation method of biological microenvironmental factors as independent osteogenic factors is further provided. The present integrated 3D printing technology realizes 3D printing of cells and hard materials synchronously by adjusting the temperature, so as to build a real sense of biomimetic bone tissue, which can be customized according to the specific defects and clinical needs of patients. In the present bone-repair functional module, the cells have high survival rate and proliferation activity on the surface of hard materials, and realize osteogenic differentiation and mineralization; after implantation, it has the dual metabolic functions of bone formation and bone resorption, promoting vascular and neurogenesis, improving elastic modulus and reducing stress shielding.

### CROSS REFERENCE TO THE RELATED APPLICATIONS

This application is based upon and claims priority to Chinese Patent Application No: 202111169004.9, filed on Sep. 30, 2021, the entire contents of which are incorporated herein by reference.

### TECHNICAL FIELD

The present invention relates to the fields of orthopedic implants and high-strength biomedical materials, advanced manufacturing and intelligent manufacturing technology. In particular, it relates to an integrated 3D bioprinting method of hard materials and cells, a preparation method and application of bone-repair function modules and bone organoids.

### BACKGROUND

A large number of people suffer from defects every year due to trauma, inflammation, tumor resection, and other reasons. However, the human body cannot regenerate and repair large critical bone defects by itself. In most cases, external surgical intervention is needed to restore the normal condition. At present, the commonly used bone graft materials in clinical practice are as follows: Autologous bone, the material taken from the patient’s own body, is the most ideal repair, but there are problems such as secondary injury, complications in multiple surgical sites and donor sites, and insufficient sources; allogeneic bone, mostly from cadaveric donations or animals, has problems with immune response, potential infection risk and medical ethics. Therefore, it is of great significance to develop new bone graft materials suitable for replacing or repairing bone defects and the corresponding preparation process.

There are two different structures in normal human bone tissue: cancellous bone and cortical bone. Cancellous bone is porous, with a porosity of 45% to 90%; cortical bone is more dense, and has a porosity of 5% to 20%. However, in both cancellous and cortical bone, interconnected pore structures are very important to promote the continuous inward growth of bone tissue. This is because interconnected pores allow nutrients and oxygen to be transported to the inside of the scaffold, promote the growth of cells and tissues towards the inner structure, and promote vascularization into the scaffold, and metabolite removal out of the scaffold.

At present, porous bone-repair scaffolds can be manufactured by a variety of methods, such as chemical/gas foaming, solvent casting, particle leaching, freeze-drying, etc., but these 3D scaffold preparation techniques have poor control over the shape, size, structure, and connectivity of the pores. Using 3D printing method to design and manufacture the scaffold can effectively solve the above problems. At the same time, the 3D printing method can also be customized according to the patient’s specific defect site, shape, and clinical needs.

In recent years, the improvement of 3D printing methods makes the 3D bioprinting technology of living cells more and more advanced. The application of biomaterials such as hydrogel to wrap cells can solve the problems of cell survival and functionalization. However, the mechanical strength of this hydrogel biomaterial is very low, far from reaching the strength requirements of orthopedic implants. Using a novel 3D bioprinting method that combine the preparation of porous bone-repair scaffolds with living cell printing has become an issue that scientists want to solve first, this technology rises a new generation of bone tissue engineering and become a research hotspot for future medicine.

At present, tissue engineering requires high mechanical properties of porous materials, which limits the selection of materials used to fabricate porous bone scaffolds. However, materials with high mechanical strength tend to have a high melting point, which makes it difficult to print synchronously with living cells. Currently, 3D printing of the existing porous scaffolds and living cells are carried out separately, independently of each other. Although hydrogels are also biomaterials with certain strength to wrap; as mentioned above, current hydrogels are difficult to achieve the mechanical strength of bone. Cells can only attach to the surface of porous scaffolds to grow, but cannot grow inside of the porous scaffolds. The preparation of biomimetic bone tissue engineering materials has not been realized in a real sense.

Therefore, one of the assessment indicators set in Project 1.6 “Technology and Equipment of Precisely 3D Printing of Multiple Cell Types” in the 2018 Project Application Guide of the Key Special Project “Additive Manufacturing and Laser Manufacturing” by the Ministry of Science and Technology of China is “to ensure that more than 85% of cells survive for at least 10 days”. Only by solving the problem of cell survival hard materials, can we play the biological function of cells to touch the goal of clinical treatment.

Therefore, the discovery and construction of in vivo osteogenic biological microenvironment are the premise of endowing the 3D bioprinted composite structure of biomaterials and cells with the function of bone-repair. Carrying out high quality work of bone formation and bone reconstruction engineering after the module are transplanted into the body will perfectly, complete the bone tissue regeneration and repair, and establish the industry standards for clinical application.

In addition, bone tissue is formed by biomineralization of bone matrix produced by osteoblasts. Its formation process is complicated and related to the physiological activities of a variety of bone cells. However, the existing bone tissue materials loaded with live cells only contain a single type of cell, which has poor ability in osteogenic differentiation and is not conducive to early osseointegration.

In view of this, the present invention is proposed.

### SUMMARY

The purpose of the invention is to provide a novel 3D bioprinting method integrating biomaterials and cells, and to prepare materials for bone defect repair by applying the method. The integrated 3D bioprinting method uses the multi-nozzle cooperative printing to realize the alternating parallel printing and layer by layer arrangement of biomaterials and cells by controlling the printing temperature of different nozzles, so as to bionic construct a bone-repair functional module with a pore connected structure.

Another purpose of the invention is to provide a bone defect repair material with living cells chimed into a porous bone-repair functional module, to create a suitable biological microenvironment through the loaded cells, and to achieve the purpose of biomimetic bone tissue in structure, composition and function.

The present invention further aims at providing the application of the aforementioned bone-repair functional modules and bone defect repair materials in the preparation of the products of hard tissue replacement and/or repair materials.

In order to solve the above technical problems and achieve the above purposes, the invention provides the following technical solutions:

First, the invention provides a 3D printing method for integrating hard materials and cells, including a method for preparing bone-repair functional modules by integrating high-strength biomedical materials and cells with synchronous 3D printing;

The high-strength biomedical material refers to the hard material with compression strength of 2 MPa and above;

The high-strength biomedical material is printed in the form of a hard material bundle, and the cell is printed in the form of a cell bundle;

The integrated three-dimensional printing method includes the use of a multi-nozzle alternately printed hard material bundle and cell bundle, so that the hard material bundle and cell bundle are arranged in parallel into layers, and then printed layer by layer into a three-dimensional structure with a hole. Between the two adjacent layers, the hard material bundle and cell bundle are perpendicular or at an angle to each other, and the three-dimensional bone-repair functional module is obtained;

The multi-nozzle comprises at least two nozzle, namely material printing nozzle and cell printing nozzle;

The cells include cells that create an osteogenic microenvironment.

The cells that create osteogenic microenvironment refer to that a certain cell overexpresses or inhibits the expression of one or more osteogenic factors by activating or inhibiting one or more cell signaling pathways, so as to exert a positive influence on the proliferation, differentiation or metabolic process of other cells related to osteogenesis in the repair tissue area. Finally, a variety of cells and their expression products together constitute a tissue microenvironment conducive to osteogenesis.

In alternative embodiments, the cells that create an osteogenic microenvironment include cells related to bone tissue formation;

Preferably, the cells that create the osteogenic microenvironment include bone marrow stromal cells, bone progenitor cells, preosteoblasts, osteoblasts, bone lining cells, osteocytes or osteoclasts a combination of at least one cell or more than two cell types;

Preferably, the cells that create the osteogenic microenvironment are bone marrow stromal cells or osteocytes or a combination of both;

Preferably, the osteocytes are activated by Wnt signaling, which are used to create an osteogenic microenvironment, promote the proliferation, osteogenic differentiation and mineralization of bone marrow stromal cells, promote the differentiation of osteoclasts, and promote the regenerative repair of bone defects;

Preferably, the Wnt signaling activation method includes the activation of carnonical Wnt/β-catenin signaling by one or more components of biomedical materials, small molecule drugs, proteins, and peptides;

Preferably, the number ratio of the Wnt signaling activated bone cells to bone marrow stromal cells is 1: (2 to 8);

Preferably, the number ratio of the Wnt signaling activated osteocytes to bone marrow stromal cells is 1:4;

Preferably, the osteocytes overexpressing at least one osteogenic biological microenvironment factor;

Preferably, the osteogenic biological microenvironment factor comprises a Delta like canonical Notch ligand 4(D114).

Among the optional embodiments, the printing method of the hard material bundle includes that a high-strength biomedical material is sequentially melted and extruded by a mechanical screw propoller to obtain a hard material bundle; and/or the printing method of the cell bundle includes a hydrogel or bioink that wraps the cell and is extruded by air pressure-driven extrusion to obtain the cell bundle.

Preferably, the melting temperature of the hard material is 30 - 200° C., and the printing temperature of the hard material bundle is 30 - 200° C.

Preferably, the printing temperature of the cell bundle is 4 to 37° C.

The integrated 3D printing refers to that in the 3D printing process, the additive process of high-strength biomedical materials and cells is carried out synchronously to form a single layer and the printing is continued for another layer, in which the printed layers are perpendicular or at an angle to the ones of the neighboring layer to obtain stacked layers as a module. The invention can realize the cooperative printing of hard material bundles with different high temperature (> 60° C.) or low temperature (< 60° C.) material bundles and low temperature (≤ 37° C.) cell bundles in the same 3D printing space by gradient adjustment of the temperature of 3D printing between 4° C. and 100° C. As a result, cells can be programmed to fit into biomaterials.

The printing speed of the 3D printing is 2 - 10 mm/s.

The printing speed is determined according to the performance of the hard material bundle and the cell bundle. The general printing speed of the hard material bundle is 2 - 5 mm/s, and that of the cell bundle is 5 - 10 mm/s.

Second, the invention provides a bone-repair functional module obtained by the printing method of any of the aforementioned implementation methods, which comprises a material unit for mechanical scaffolds, a cell unit for osteogenic function and a channel; the volume ratio of the material unit and the cell unit in the osteoid hard tissue module is 1:0.5 - 2, and the porosity of the osteoid hard tissue module is 20% - 80%.

The cells in the bone-repair functional module not only adhere to the surface, but also integrate into the interior of the module. At the same time, for the purpose of biomimetic normal bone tissue, it can also realize different distribution modes of bone-repair functional modules and cell units, as well as the porosity of bone-repair functional modules.

Preferably, the channel includes one or more combinations of any channels, such as a through hole, a buried hole, or a blind hole.

Preferably, the printing method of the pore channel includes the separation of adjacent hard material bundles and cell bundles, or the printing of the pore forming material, and after the printing is completed, the pore forming material is removed to form the pore channel.

Preferably, the channel forming material comprises a sacrificial material.

Preferably, the sacrificial material includes Pluronic F127.

The channels can directly control the printing platform to form a three-dimensional channel structure after accurately printing the material bundle and the cell bundle, or use the channel forming material, such as printing the sacrificial material Pluronic F127 into a bundle, washing it out in solution after printing, and its original space position is the channel structure in the functional module.

As a channel for nutrient component transport and metabolite removal, and the space between the collagen secreted by osteoblasts and the natural and induced blood vessels after transplantation into the body, the distribution mode is also uniformly arranged in the bone-repair functional module for the purpose of biomimetic bone tissue structure.

In an alternative embodiment, the material unit includes a composite polymer containing hydroxyapatite.

Preferably, the particle size of the hydroxyapatite is nanometer.

Hydroxyapatite is an important mineral component in bone tissue, and it is also the most widely used inorganic material with good bioactivity and biocompatibility in biomaterials. The addition of hydroxyapatite to the composite polymer scaffold can significantly improve the biological properties of the scaffold, and improve the mechanical properties such as hardness, compressive strength and wear resistance of the scaffold, and the improvement effect is enhanced with the decrease of the particle size of hydroxyapatite.

Preferably, the polymer material used in the composite polymer includes one or more combinations of polycaprolactone and its derived co-polymer.

Preferably, the mass ratio of nano-hydroxyapatite to polymer material is 1:(4 - 9).

Preferably, the mass ratio of nano-hydroxyapatite to polymer material is 1:9.

The composite polymer scaffold material provided by the invention has a low melting point and can meet the demand of simultaneous printing with cells. At the same time, the addition of hydroxyapatite also makes it with a higher strength, which can make up for the weakening effect of cell unit chimerism on the mechanical strength of bone-repair function module.

In alternative embodiments, the cell unit includes a hydrogel or bioink that encapsulates the cell. The density of the cells in bioink or hydrogel ranges from 1×10⁵ to 1×10⁷ cells/ml, preferably 1 × 10⁶ cells/ml.

Preferably, the bioink or hydrogel contains solidified molecules.

Preferably, the solidified molecule includes methylacrylylated gelatin.

Osteocytes account for more than 90% of normal bone tissue cells and play an important role in bone development and maintenance of bone homeostasis. Compared with wild osteocytes, osteocytes activated by Wnt signaling not only expanded stem cells, but also promoted osteogenic differentiation and activated osteoclast function. Therefore, using Wnt to activate osteocytes combined with bioink can achieve the purpose of building a biological microenvironment, which can promote the proliferation and osteogenic differentiation of bone marrow stromal cells, activate the function of osteoclasts at the defect site, and promote bone regeneration to repair bone defects.

The hydrogel or bioink includes a cell growth medium containing penicillin-streptomycin and/or fetal bovine serum, optionally growth medium including α-MEM medium. For example, α-MEM medium containing 10% FBS, as a percentage by volume, contained 50 to 100 U/mL penicillin and 50 to 100 µg/mL streptomycin, as a percentage by mass. Fetal bovine serum can be 8% to 20%. Optionally, the bioink or hydrogel also contains a solidifying molecule, and optionally, the solidifying molecule includes methylacrylylated gelatin.

The hydrogel or bioink provided by the invention can be in a stable form for more than 28 days in vitro after cross-linking molding; the cell survival rate was higher than 89%, and the cell proliferation activity was high.

The bioink provided by the invention not only contains the nutrients required for cell proliferation and differentiation, but also plays a protective role for cell activity in the preparation process of 3D printing to avoid the reduction of cell viability or even inactivation due to shear force or heat.

Third, the present invention provides a repair material of bone defects, including bone-repair functional modules and bone organoids. The preparation method of the bone defect repair material includes the bone-repair functional module prepared by the integrated three-dimensional printing method mentioned in any of the foregoing implementation methods, or the bone-repair functional module mentioned in any of the foregoing implementation methods. Bone defect repair materials were obtained after in vitro culture.

Preferably, the in vitro culture conditions include a bone-repair functional module cultured for 7 to 30 days in cell growth medium in an incubator or bioreactor with a volume ratio of 5% carbon dioxide and a temperature of 37° C.

Preferably, the cell medium is cell growth medium, and the bone defect repair material obtained after culture is the functional module of bone-repair.

Preferably, the bone-repair functional module is cultured with osteogenic differentiation medium, and the bone defect repair materials obtained is mineralized bone organoid.

Preferably, the osteogenic differentiation medium contains dexamethasone, vitamin C, and sodium βglycerol phosphate.

In an alternative embodiment, the bone defect repair materials include bone-repair functional modules in which osteocytes overexpressing osteogenic biological microenvironmental factor(s). The osteocytes overexpress osteogenic biological microenvironmental factors Osteocytes in the functional module of bone-repair overexpress at least one osteogenic biological microenvironmental factor.

Preferably, the osteogenic biological microenvironmental factors include the D114.

In an alternative embodiment, the D114 acts as a Notch signaling ligand to activate the classical Notch signaling pathway in target cells.

Preferably, the target cells include at least one of bone progenitor cells, preosteoblasts, osteoblasts, bone lining cells, bone marrow stromal cells, and/or osteoclast and its precursors.

D114 overexpressed by osteocytes is a Notch signaling ligand and a Notch signaling provider. Through Notch signaling receptors in target cells, it activates classical Notch signaling in target cells dependent on Notch signaling transcription factor RBPjκ, promoting the survival, proliferation, osteogenic differentiation and mineralization of target cells in vitro. The target cells containing Notch signaling receptors include bone progenitor cells, preosteoblasts, osteoblasts, bone lining cells, bone marrow stromal cells, and/or osteoclast and its precursors. After the target cells are activated, the intracellular fragment NICD of Notch receptor, the Notch signaling transmitter, is generated. NICD enters the nucleus, thereby activating the Notch signaling transcription factor RBPjκ, initiating the transcription and expression of Notch signaling Hes/Hey family and other target genes, thereby promoting the proliferation, differentiation and rapid bone formation of target cells. It also promotes endothelial cells to form blood vessels.

Conventional Notch signaling activation methods include the use of biomedical materials, small molecule drugs, proteins, peptides and one or more components to activate classical Notch signaling in target cells.

Fourth, the present invention provides a bone-repair functional module prepared by the three-dimensional printing method of the integration of hard materials and cells mentioned in any of the aforementioned implementation methods, the bone-repair functional module mentioned in any of the foregoing implementation methods, and the application of bone defect repair materials mentioned in any of the foregoing implementation methods in the preparation of products for tissue replacement and/or repair materials;

When the product of the tissue replacement and/or repair material is cultured in vitro, the cells on the surface of the product of the tissue replacement and/or repair material have high survival rate and proliferation activity, and can successfully achieve osteogenic differentiation and mineralization; after implantation in animals, it has dual metabolic functions of bone formation and bone resorption, as well as vasculogenic and neurogenic functions;

The tissue includes a hard tissue structure or a skeletal structure in soft tissue.

The invention has the following beneficial effects:

The invention provides an integrated three-dimensional printing method for hard materials and cells. In this method, the hard material bundle and the cell bundle are alternately printed by multiple nozzles, realizing the integration of hard material and cell three-dimensional alternatively, layer-by-layer printing, so as to complete the real sense of bionic bone tissue construction of bone-repair functional modules. It can be customized according to patient specific defects and specific clinical needs.

In the bone-repair functional module or bone organoid obtained by the above preparation method, the preliminary integration of cells and biomaterials is realized, and the preliminary osteogenic differentiation and mineralization are formed, which has certain biological functions. At the same time, it reduces the early stress shielding effect after implantation of bone defect repair materials, and further promotes the early osseointegration compared with the direct implantation of bone-repair functional modules. It is especially suitable for the preparation of hard tissue replacement or repair materials.

Compared with the prior art, the special feature of the invention lies in that the 3D printed module provided by the invention can form the bone-repair functional module and bone organoid. It has the basic metabolic functions of bone formation (osteoblast function) and bone resorption (osteoclast function), as well as the formation of a large number of blood vessels to ensure organ nutrient supply and efficient transport of metabolites. In addition, after in vitro culture, the above-mentioned bone-repair functional modules can achieve osteogenic differentiation and mineralization into bone organoids in vitro, which can be directly used for bone defect repair under special conditions.

### BRIEFT DESCRIPTION OF THE DRAWINGS

In order to more clearly describe the specific implementation mode of the invention or the technical scheme in the prior art, the following is a brief introduction to the supplementary drawings required in the description of the specific implementation mode or prior art. It is obvious that the drawings described below are some embodiments of the invention and that other drawings may be obtained from them without creative effort by ordinary technicians in the field.

FIGS. 1A-1C are schematic diagrams of the appearance and internal structure of the printer provided in the specific embodiment of the invention;

FIGS. 2A1-2C are an example of a hard tissue repair material that can be used for printing by the printing method provided by the invention;

FIGS. 3A-3F are the schematic diagram of the experimental process of example 1;

FIG. 4 is a qualitative test result of alkaline phosphatase activity in example 1, comparative example 1 and comparative example 2;

FIG. 5 is a qualitative test result of alkaline phosphatase activity in example 4, comparative example 4 and comparative example 6;

FIG. 6 is a quantitative test result of alkaline phosphatase activity in example 1, comparative example 1 and comparative example 2;

FIG. 7 is a quantitative test result of alkaline phosphatase activity in example 4, comparative example 4 and comparative example 6;

FIG. 8 is a comparison of expression results of osteogenesis related genes in example 1, comparative example 1 and comparative example 2;

FIG. 9 is a comparison of expression results of osteogenesis related genes in example 4, comparative example 4 and comparative example 6;

FIGS. 10A-10G are a comparison of the performance of the bone-repair functional module obtained in vitro in example 1;

FIGS. 11A-11B are a comparison of cell proliferation activities of examples 1 to 3;

FIGS. 12A-12B are a comparison of cell proliferation activities of example 1, comparative example 1 and comparative example 2;

FIGS. 13A-13D are a comparison of cell survival effect and osteogenic differentiation degree in example 1 and example 5;

FIGS. 14A-14B are a comparison before and after implantation of mouse model example 1 and comparative examples 1 and 2;

FIG. 15 shows the results of micro CT examination of the repair of parietal bone defects in example 1 and comparative examples 1 and 2 after implantation of a mouse model;

FIGS. 16A-16H are a comparison of bone histomorphology of parietal bone defect repair in example 1 and comparative examples 1 and 2 after implantation of a mouse model;

FIGS. 17A-17B are a comparison of bone matrix analysis of parietal bone defect repair in example 1 and comparative examples 1 and 2 after implantation of a mouse model;

FIGS. 18A-18B are a comparison of bone formation ability of parietal bone defect repair in example 1 and comparative examples 1 and 2 after implantation of a mouse model;

FIGS. 19A-19D are a comparison of osteoclast staining in the repair of parietal bone defects in example 1 and comparative examples 1 and 2 after implantation of a mouse model;

FIGS. 20A-20D are a comparison of the angiogenic ability of example 1 and comparative examples 1 and 2 after implantation of a mouse model;

FIG. 21 is a comparison of peripheral nerve formation ability of parietal bone defect repair in example 1 and comparative examples 1 and 2 after implantation of a mouse model;

FIGS. 22A-22B are a comparison of in vitro mineralization detection results of example 1 and comparative examples 1 and 2;

FIGS. 23A-23B are a comparison of in vitro mineralization detection results of example 4 and comparative examples 4 and 6;

FIGS. 24A-24D are a comparison of the measurement results of the relationship between the osteogenic differentiation function of example 4 and comparative examples 4 and 6 and the Notch signaling of BMSCs inhibited by drugs;

FIGS. 25A-25D show the comparison of the relationship between Notch signaling and osteogenic differentiation in BMSC primary cells of RBPjκ^{f/f} mice before and after RBPjκ gene knockout;

FIGS. 26A-26C show the comparison of the relationship between Notch signaling and osteogenic differentiation of BMSC primary cells before and after RBPjκ gene knockdown in example 4 and comparative example 6;

FIGS. 27A-27B are a comparison of the measurement results of the angiogenic function in example 4 and comparative examples 4 and 6.

### DETAILED DESCRIPTION OF THE EMBODIMENTS

In order to make clear the purpose, technical solution and advantages of the embodiment of the invention, the technical solution of the embodiment of the invention is clearly and completely described in the following in combination with the attached drawings in the embodiment of the invention. It is obvious that the described embodiment is a part of the embodiment of the invention, but not all of the embodiment. The components of embodiments of the invention described and shown herein may be laid out and designed in various configurations.

Therefore, the following detailed descriptions of embodiments of the invention provided in the supplementary drawings are not intended to limit the scope of the invention for which protection is claimed, but merely represent selected embodiments of the invention. Based on the embodiments in the present invention, all other embodiments obtained by ordinary technicians in the field without creative labour fall within the scope of protection of the present invention.

It should be noted that similar labels and letters denote similar terms in the appendixes below, so that once an item is defined in one appendix it does not need to be further defined and explained in subsequent appendixes.

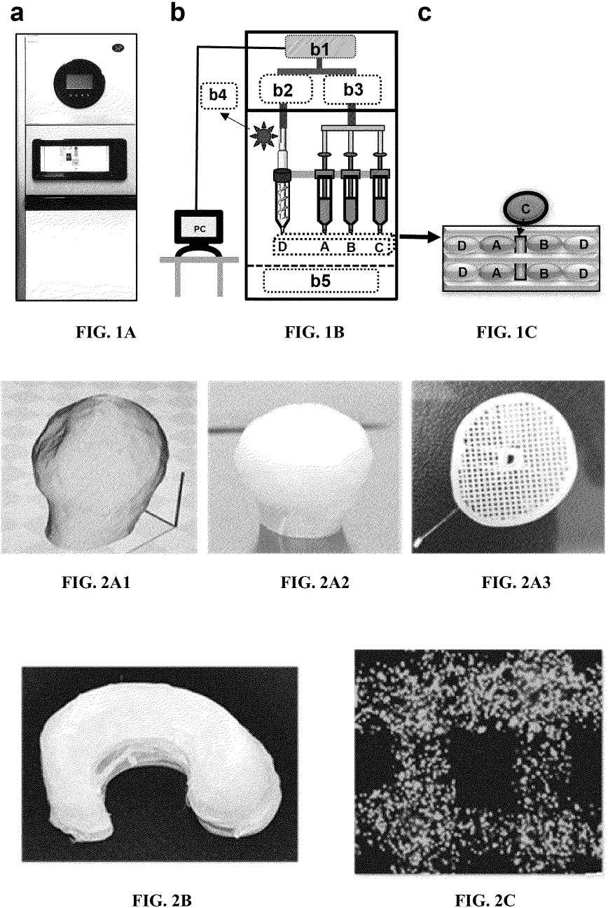

In a concrete implementation, the invention provides a three-dimensional bioprinter with integrated hard material and cell as shown in FIGS. 1A-1C, in which FIG. 1A is the appearance diagram of the printer and FIG. 1B is the internal structure diagram controlled by the program. Wherein b1 is a three-axis workbench, and b2 is a screw extrusion unit. A screw extrusion device is arranged in the connected hard material bundle printing nozzle, and a heating unit is arranged outside the screw extrusion device to print the hard material bundle (D). b3 is an air-pressure drive unit, and the connected cell bundle printing nozzle is used to print cell bundle (A and B) or sacrificial material (C). b4 is the light source, including illumination light source, sterilization light source or other functional light source. b5 is the temperature control system, under the printing platform, used to adjust the temperature of the printing platform when printing different materials. FIG. 1C is the schematic diagram of hard material bundle (D), cell bundle (A and B) and sacrificial material bundle (C) after printing, in which sacrificial material bundle (C) is washed away by the medium to obtain the corresponding channel.

The above printer can print a variety of hard tissue repair materials, and is suitable for the preparation of long bones including femur, tibia, humerus, ulna, radius, meniscus, and parietal bone etc., as shown in FIGS. 2A1-2C. Wherein FIG. 2A1-2A3 are the femoral head material printed by the above printer, Wherein FIG. 2A1 is the 3D modeling example of the femoral head, FIG. 2A2 is the physical photo of the femoral head material printed by the above printer, FIG. 2A3 is the cross-sectional diagram of A2, showing the uniformly printed material bundle. FIG. 2B is the real picture of meniscus printed by the above printer. FIG. 2C is the detection result of printed cells expressing GFP under fluorescence microscope. It can be seen that living cells are evenly distributed in the hydrogel and grow well. It indicates that the living cells printed by the printer can achieve normal growth.

Some embodiments of the present invention are described in detail in the light of the attached drawings. The following embodiments and the characteristics in the embodiments may be combined without conflict.

### Example 1

This embodiment is aimed at the wild-type C57BL/6 mouse parietal bone defect model with a diameter of 4.5 mm, and provides a bone-repair functional module prepared by integrated 3D printing of high-strength biomedical materials and cells. The equipment used in the integrated 3D printing method is shown in FIGS. 1A-1C. The printing method includes the following steps:

### 1.1 Preparation of Hard Materials

18 g polycaprolactone and 2 g nano-hydroxyapatite were weighed and mixed into the screw extrusion device. Firstly, it was heated to 95° C., so that the polycaprolactone and hydroxyapatite became mobile phase, and the hard material was obtained upon cooling.

### 1.2 Configuration of Hydrogels

A concentration of 20% (w/v) of GelMA was added to α-MEM medium containing 10% FBS by volume and a final concentration of 50 U/mL penicillin and 50 µg/mL streptomycin under aseptic conditions. LAP concentration of 20% (v/v) was added, and the mixture was stirred at 37° C. until no precipitate was found. After filtration with 0.22 µm filter membrane, glycerol concentration of 10% (v/v) was added, and the mixture was stirred at 37° C. for 1 h. The final concentration of the methylacrylylated gelatin after addition of an equal volume of cell suspension is 100 mg/mL.

### 1.3 Configuration of Cell Suspension

(1) The supernatants of primary bone marrow stromal cells and Wnt signaling activated osteocytes in good culture state were removed and washed twice with PBS.
(2) Then 1 mL 0.25% trypsin was added and digested for 5 min at 37° C. in a cell incubator.
(3) The cells were gently blown, and the cell suspension was collected and centrifuged at 850 rpm for 5 min.
(4) Remove the supernatant. Appropriate amount of cell medium was added and resuspended and counted for later use.
(5) According to the ratio of WNT-activated osteocytes to bone marrow stromal cells (1:4), the cell suspension with cell density of 2.0× 10⁶ cells /mL was prepared for use.

### 1.4 Preparation of Cell Printing Solution

A cell printing solution with a cell density of 1×10⁶ cells /mL was obtained by evenly mixing 1 mL hydrogel and 1 mL cell suspension, which was stored at 37° C. and precooled at 4° C. for 10 minutes before printing.

### 1.5 3D Printing of Bone-Repair Functional Modules

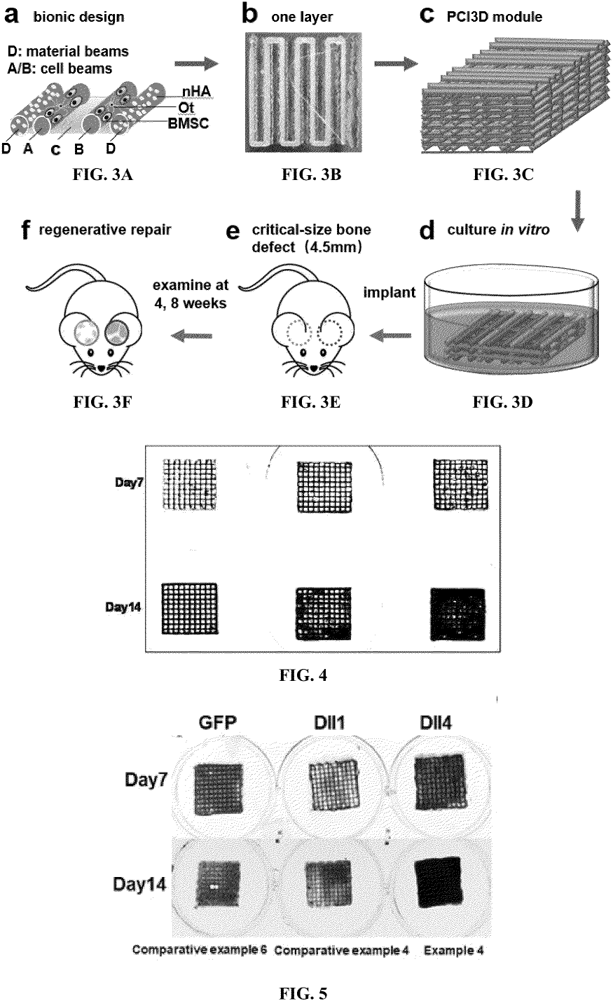

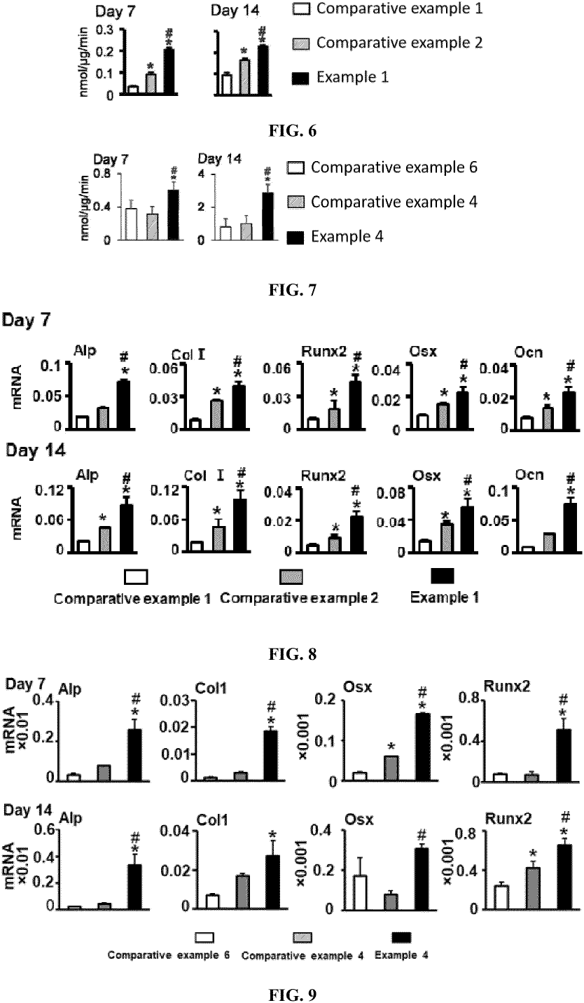

(1) Micro-CT was used to scan the bone defect area of mice, and Mimics software was used to process the CT scan results. The three-dimensional model of the bone defect area was established and stored as. STL format file.
(2) Import the above files into the integrated 3D bio-printer. The structure diagram of the integrated 3D bio-printer is shown in FIGS. 1A-1C. According to the material strength requirements, the experiment needs to build scaffolds with mechanical and mechanical support functions, provide space for cell growth, build functional module models, and save them as program files of Gcode.
(3) Set print-related parameters, including temperature, speed, and height. Open the Gcode file and start printing. The printing temperature of the hard material bundle is 80° C. and that of the cell bundle is 27° C.
(4) After cell printing, the light source (b4) in FIGS. 1A-1C was adjusted to a blue light lamp, and blue light cross-linking was performed on the hydrogel in the obtained bone-repair functional module. The cross-linking result is shown in B in FIGS. 3A-3F. It can be seen that a layer printed in this embodiment shows that the hard material is arranged evenly in bundles, and the cells in the bundle grow well and are distributed evenly (as shown in FIG. 2C).
(5) The bone-repair functional module was placed in a 5% CO₂ incubator at 37° C. and cultured with growth medium for 14 days to obtain the bone-repair functional module (as shown in FIG. 3D).
(6) The bone-repair functional module obtained in step (5) was implanted into a wild-type C57BL/mouse model with a parietal diameter of 4.5 mm critical bone defect, and serial tests were performed at 4 and 8 weeks after implantation.

The experimental process of this embodiment is shown in FIGS. 3A-3F, in which FIG. 3A is the alternating printing design of hard material bundle and cell bundle, FIG. 3B is the one-layer physical picture of hard material bundle and cell bundle obtained by 3D printing, and FIG. 3C is the schematic diagram of bone-repair functional module obtained by cumulative printing between layers. After the printed bone-repair functional module was cultured in the cell culture plate (FIG. 3D), the obtained bone-repair functional module was implanted in the mouse parietal critical bone defect (> 4 mm), and its repair function was evaluated at the fourth and eighth week after implantation (FIGS. 3E-3F).

### Example 2

The difference between this comparative example and example 1 is that the cell density in this comparative example is 1 × 10⁵ mL.

### Example 3

The difference between this comparative example and example 1 is that the cell density in this comparative example is 1 × 10⁷ mL.

### Example 4

This example is different from example 1 in that Wnt activated bone cells are replaced with bone cells overexpressing Dll4. No sacrificial material was used.

### Example 5

The difference between this example and example 1 is that methacrylated gelatin is not added to the hydrogel.

### Comparative Example 1

The difference between this comparative example and example 1 is that only a single bone marrow stromal cell was selected in this comparative example and was recorded as a control group.

### Comparative Example 2

The difference between this comparative example and example 1 is that in this comparative example, bone marrow stromal cells and wild bone cells that do not activate Wnt signalings are selected as the wild group.

### Comparative Example 3

The difference between this comparative example and example 1 is that hydroxyapatite is not added to the precursor liquid of the material unit.

### Comparative Example 4

This comparative example is different from example 4 in that Wnt activated bone cells are replaced with bone cells overexpressing Dll1.

### Comparative Example 5

This comparative example is different from example 4 in that Wnt activated bone cells are replaced with bone cells overexpressing Dll3.

### Comparative Example 6

The difference between this comparative example and comparative example 4 is that the bone cells were also transfected with recombinant lentivirus overexpressing GFP gene.

Osteocytes overexpressing Dll1, Dll3, D114 or GFP gene were constructed as follows:

Recombinant lentiviruses containing Dll1, Dll3, D114 or GFP genes were transfected into MLO-Y4 cells at a complex number of infection (MOI) of 100 and polybrene (7 µg/mL) respectively. The efficiency of each lentivirus infection was determined by the intensity of GFP fluorescence after 72 h of culture. Untransfected cells were then eliminated by screening with medium containing 0.5 µg/mL puromycin.

### Experimental Example

The following detection methods were used to detect the bone like hard tissue module, bone-repair functional module or bone like organ obtained in the above examples and comparative examples.

### 1. Qualitative Detection of Alkaline Phosphatase Activity

After the bone-repair functional modules obtained in examples and comparative examples were cultured in the incubator for 7 days and 14 days, the alkaline phosphatase activity of the cells in the cultured bone-repair functional module was qualitatively detected by staining. The specific detection method was referenced in “Tu X et al(2007)Noncanonical Wnt signaling through G protein - linked PKCδ activation promotes bone formation. Dev Cell 12(1): 113-27”.

Example 1, comparative example 1 and comparative example 2 were qualitatively tested for alkaline phosphatase activity, and the results are shown in FIG. 4. It can be seen from FIG. 4 that after 7 days and 14 days of culture, the qualitative measurement results of alkaline phosphatase activity of the bone-repair functional module constructed by Wnt activated bone cells in example 1 (right in FIG. 4) were higher than those in comparative examples 1 and 2, which proved that Wnt signaling activates the printing combination of osteocytes and bone marrow stromal cells. Compared with wild-type osteocytes and/or bone marrow stromal cells, it is more effective in promoting osteogenic differentiation.

Example 4, comparative example 4 and comparative example 6 were tested qualitatively for the above alkaline phosphatase activity, and the results are shown in FIG. 5. It can be seen from FIG. 5 that after 7 days and 14 days of culture, the alkaline phosphatase activity measurement results in the bone-repair functional module of bone cell D114 in example 4 (right in FIG. 5) were higher than those in comparative example 4 and comparative example 6, which proved the printing combination of osteocytes overexpressing D114 and bone marrow stromal cells. Compared with the print combination of osteocytes overexpressing GFP or Dll3 and bone marrow stromal cells in the control group, it can promote osteogenic differentiation more.

### 2. Quantitative Determination of Alkaline Phosphatase Activity

The bone-repair functional modules of example 1 and comparative examples 1 and 2 were harvested after 7 and 14 days of culture in vitro respectively. The cells in the module were lysed with Biyuntian lysate (P0013J, without inhibitor), and the alkaline phosphatase activity was quantitatively detected with the alkaline phosphatase activity quantitative detection kit (Biyuntian P0321S) after cell lysis. Methods Refer to “Tu X etal (2007)Noncanonical Wnt signaling through G protein - linked PKCδactivation promotes bone formation. Dev Cell12(1):113-27”.

Example 1, comparative example 1 and comparative example 2 were cultured in vitro and the results were as shown in FIG. 6. It can be seen from FIG. 6 that after 7 days and 14 days of culture, the quantitative detection results of alkaline phosphatase activity obtained in example 1 were significantly higher than those in Comparative Example 1 and comparative example 2, which proved that Wnt signaling activates the printing combination of osteocytes and bone marrow stromal cells. Compared with wild-type osteocytes and/or bone marrow stromal cells, it is more effective in promoting osteogenic differentiation.

Example 4, comparative example 4 and comparative example 6 were cultured in vitro and the results were as shown in FIG. 7. It can be seen from FIG. 7 that after 7 days and 14 days of culture, the quantitative detection results of alkaline phosphatase activity obtained in example 4 were significantly higher than those in comparative example 4 and comparative example 6, which proved the printing combination of osteocytes overexpressing D114 and bone marrow stromal cells. Compared with the printing combination of osteocytes overexpressing GFP or Dll3 and bone marrow stromal cells in the control group, it has a more effective effect on promoting osteogenic differentiation.

### 3. RNA Extraction and Determination of Gene Expression Levels

The bone-repair functional modules obtained in examples and comparative examples were cultured in a cell incubator or bioreactor for 7 days and 14 days, and the obtained cultured cells were digested to detect the expression level of the gene encoding the osteoblast marker protein expressed in the cells. For the specific detection method, please refer to the literature “Tu X etal(2015) Osteocytes mediate the anabolic actions of canonical Wnt/β-catenin signaling in bone. Proc Natl AcadSciUSA.112(5): E478 - 86”.

The above tests were performed on example 1, comparative example 1 and comparative example 2, and the test results are shown in FIG. 8. As can be seen from FIG. 8, by detecting the mRNA of five genes related to osteogenic differentiation, including ALP, it is further proved that the bone-repair functional module provided in example 1 has the function of promoting osteogenic differentiation

The above tests were performed on example 4, comparative example 4 and comparative example 6, and the test results are shown in FIG. 9. It can be seen from FIG. 9 that the expression of osteoblast marker genes in example 4 is much higher than that in comparative example 4 and comparative example 6, which proves that the bone-repair functional module provided in example 4 has a good function of promoting osteogenic differentiation.

### 4. Performance Characterization of Bone-Repair Functional Modules

The bone-repair functional modules cultured in vitro in example 1 and comparative example 3 were characterized and analyzed. For specific performance characterization and testing methods, see the literature “Ma et al (2019) Three-dimensional printing of biodegradable piperazine-based polyurethane-urea scaffolds with enhanced osteogenesis for bone regeneration. ACS Applied Materials & Interfaces, 11: 9415 - 24”.

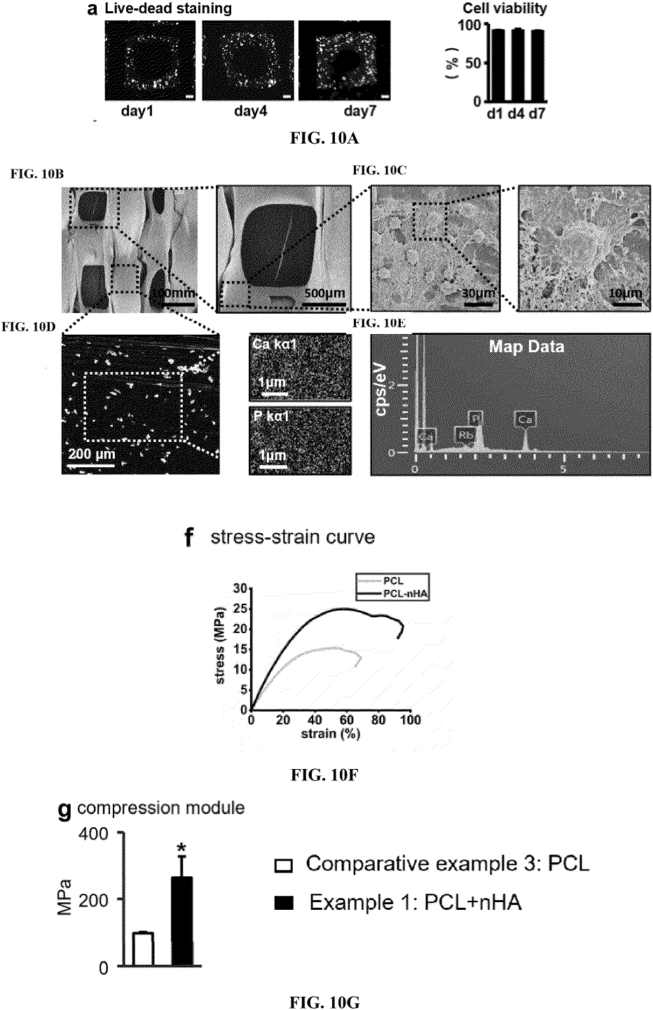

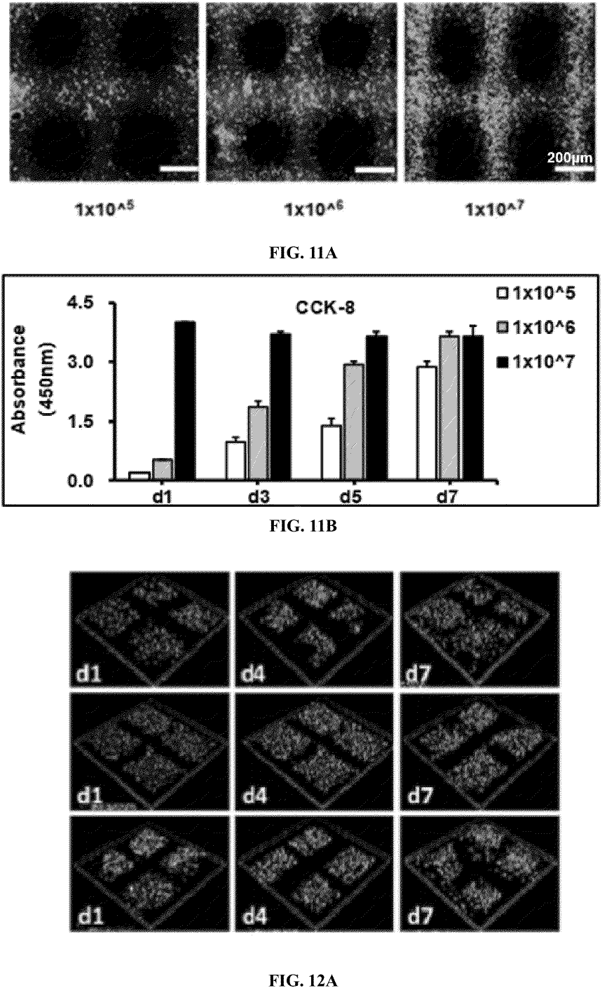

The survival rates of cells on the surface of the bone-repair functional module of Example 1 were as high as 90.0%, 91.3% and 91.9% on day 1, 4 and 7 respectively, as measured by live and dead cell staining (FIG. 10A); scanning electron microscopy showed that the functional module of bone-repair in Example 1 had a solidifying structure stably built with hard materials and a firmly formed channel (FIG. 10B). Cells adhere to hard surfaces, grow uniformly, and secrete matrix (FIG. 10C). The results of atomic force microscopy spectroscopy showed that the polycaprolactone and nano-hydroxyapatite composite had the distribution characteristics of hydroxyapatite of natural organic bone (FIGS. 10D-10E). According to the national standard (GB/T3356-2104) test method for bending properties of directional fiber reinforced polymer matrix composites, it was measured that the addition of nano-hydroxyapatite to polycaprolactone greatly improved the elastic modulus, yield force, maximum force and energy absorption of polymer materials (FIG. 10F). The elastic modulus of the bone-repair functional material provided in Comparative Example 3 was 6.2 MPa, and the elastic modulus of the bone-repair functional material obtained in example 1 after adding nano-hydroxyapatite was increased by 1.8 times to 11.3 MPa (FIG. 10G).

### 5. Effect of Cell Proliferation Activity

After the bone-repair functional modules obtained from examples 1-3 were cultured in vitro for 1 day, 3 days, 5 days and 7 days, the proliferation activity of the cells was quantitatively detected by CCK-8 method. For specific detection methods, see the literature “Ma et al (2019) Three-dimensional printing of biodegradable piperazine -based polyurethane-urea scaffolds with enhanced osteogenesis for bone regeneration. ACS Applied Materials & Interfaces, 11: 9415-24”.

Examples 1 to 3 are printed cells expressing GFP at different concentrations, and the measurement results are shown in FIGS. 11A-11B. FIG. 11A is the picture taken by confocal fluorescence microscope after one day of culture of the bone-repair functional modules of example 1-3. The fluorescently labelled cells reflect the number of viable cells, which is directly proportional to the concentration of printed cells. FIG. 11B is the detection result of CCK-8 in the modules of examples 1 to 3. It can be seen that the cells in the bone-repair functional modules of examples 1 and 2 exhibit linear proliferation, and the cell proliferation activity of example 3 hardly changes.

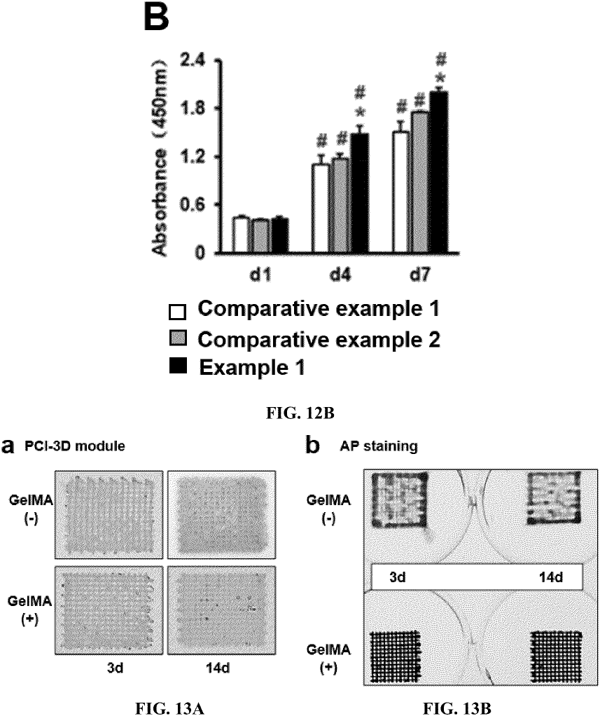

The above detection was performed on example 1, comparative example 1 and comparative example 2, and the detection results are shown in FIGS. 12A-12B. FIG. 12A is the fluorescence microscope shot after 7 days of culture. FIG. 12B is the CCK-8 detection result (* indicates the correlation with comparative example 1 p < 0.05, # indicates the correlation with comparative example 2 P < 0.05). As can be seen from FIGS. 12A-12B, cells in each functional module showed good proliferation activity. Wherein the cell proliferation activity of example 1 was significantly increased; It is suggested that Wnt signaling activation of osteocytes significantly promoted the proliferation of bone marrow stromal cells, while wild osteocytes did not show the activity of promoting the proliferation of bone marrow stromal cells.

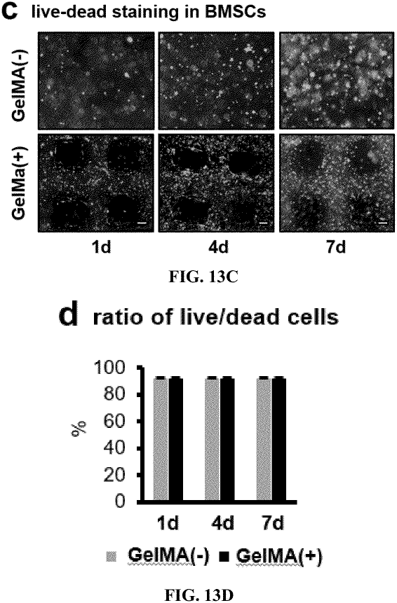

The above tests were carried out for example 1 and example 5, and the test results are shown in FIGS. 13A-13D. FIG. 13A is the physical picture of the bone-repair functional module at 3 and 14 days of culture, FIG. 13B is the qualitative detection result of alkaline phosphatase activity, and FIG. 13C and FIG. 13D are the fluorescence imaging detection results of live and dead staining of cells. It can be seen from the figure that the cells of example 1 uniformly grow, proliferate and osteogenic differentiation on the whole module, but the osteogenic differentiation of the cells of comparative example 5 is greatly reduced, and only strong osteogenic differentiation occurs around the module. Although the cells of comparative example 5 grow well and grow full of modules, the cell survival rate is as high as 92.1%, which is equivalent to that of example 1. It is fully proved that the GelMA hydrogel used is suitable for the bone-repair functional module of the invention and improves the osteogenic performance.

### 6. Construction of Mouse Parietal Defect Model and Functional Evaluation of Bone-Repair Functional Module After Implantation

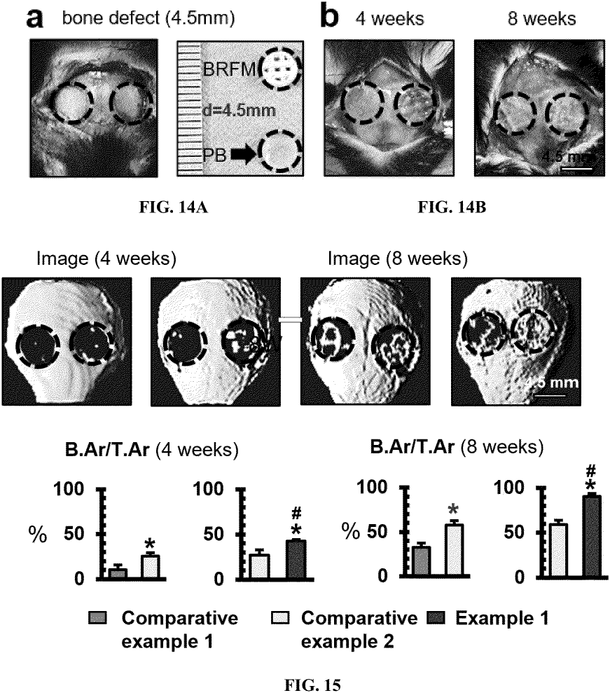

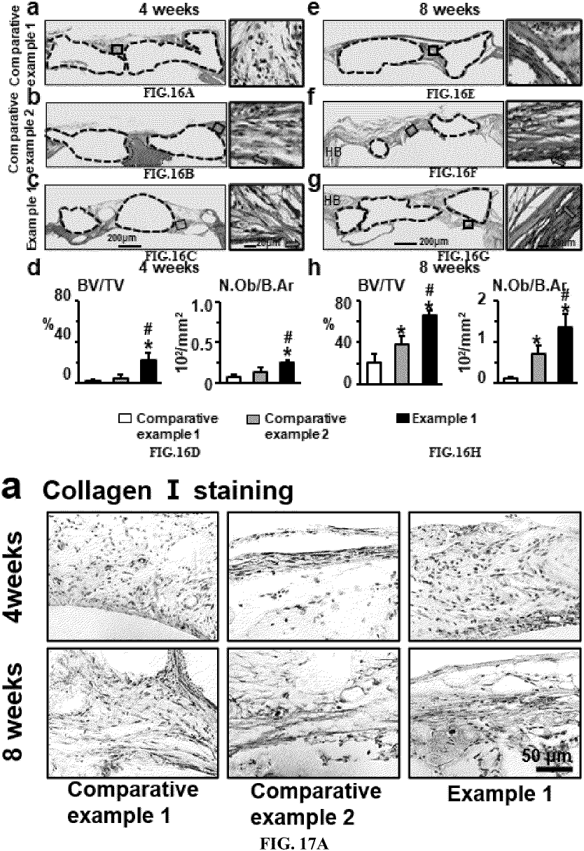

The bone-repair functional modules provided in example 1, comparative example 1 and comparative example 2 were implanted and tested as follows. For the specific detection method, refer to “Li et al (2018) Tissue-engineered bone immobilized with human adipose stem cells -derived exosomes promotes bone regeneration. ACS Appl Mater Interfaces. 10(6): 5240 - 54”.
(1)Anaesthesia: Mice were anesthetized with 3.6% chloral hydrate (Sigma-Aldrich) by intraperitoneal injection (8 µL/g);
(2) Positioning: Fixed using stereoscopic positioning device (Stoelting, Wood Dale, IL, USA);
(3) Disinfection: Use a razor to shave the hair and iodophor to disinfect the skin;
(4) Incision: a midline scalp incision was made using an 11Swann scalpel, with the skin, fascia, and parietal bone exposed in sequence;
(5) Drilling: Drill the bone block with an electric dental trephine (4.5 mm). When the bone block is to be completely removed, gently pick the bone block with the syringe needle and remove it.
(6) Implantation: the bone-repair functional module with the same shape and size is transplanted to the defect;
(7) Fixation: The module is fixed to the surrounding bone tissue with surgical sutures. The mouse model before and after implantation was compared as shown in FIGS. 14A-14B. FIG. 14A shows the preoperative implantation position, bone-repair functional module and its diameter (4.5 mm). FIG. 14B shows the comparison of physical images of implants 4 weeks and 8 weeks after implantation;
(8) Disinfection suture: Suture the skin with surgical sutures to disinfect the incision;
(9) Observation: After the mice woke up, put them back in the cage and observe them the next day.
(10) At 4 and 8 weeks after surgery, bone healing was measured as follows:

### a. Evaluation of Bone Defect Repair Function -Micro-CT Detection

Micro CT was performed on the mice at 4 and 8 weeks after operation, and the bone mass was analyzed according to the scanning results. The results are shown in FIG. 15. In the figure, the green dotted circle represents comparative example 1; the yellow dotted circle represents comparative example 2; the red dotted circle represents example 1. It can be seen that the amount of bone generated in the bone-repair functional module provided in embodiment 1 of the present invention is the most at 4 weeks and 8 weeks after the operation, and at the 8th week. In comparative Example 1, the amount of new bone formation was the least, and there was almost no bone formation at 4 weeks. This indicates that the functional module of bone-repair has a good osteogenic function in vivo and can repair critical bone defects that cannot be repaired by itself.

### b. Evaluation of Bone Defect Repair Function - Bone Histomorphology Analysis

The bone tissue was fixed, decalcified, and embedded in paraffin, then tissue sections and H.E. staining were performed. H.E. staining can distinguish chromatin, cytoplasm, extracellular matrix, and cartilage matrix by color, so as to carry out statistical analysis of new bone histomorphology, bone mass, and bone cell number, and the results are shown in FIGS. 16A-16H. FIGS. 16A-16C and FIGS. 16E-16G are H.E. staining images of the longitudinal section of the parietal defect, the dashed line is the hard material area, and the green and blue lines are the new bone generated at 4 and 8 weeks after the operation respectively, and the new bone from the amplified material. Osteoblasts can be seen after amplification (green arrow). FIG. 16D and FIG. 16H are the measurement results, showing the analysis results of bone mass (BV/TV) and osteoblast number. It can be seen that the number of osteoblasts and the bone mass are significantly higher than those of Comparative Example 1 and comparative example 2 at 4 or 8 weeks after the implantation of the bone-repair functional module with Wnt activated bone cells.

### c. Evaluation of Bone Defect Repair Function - Analysis of Bone Matrix Collagen

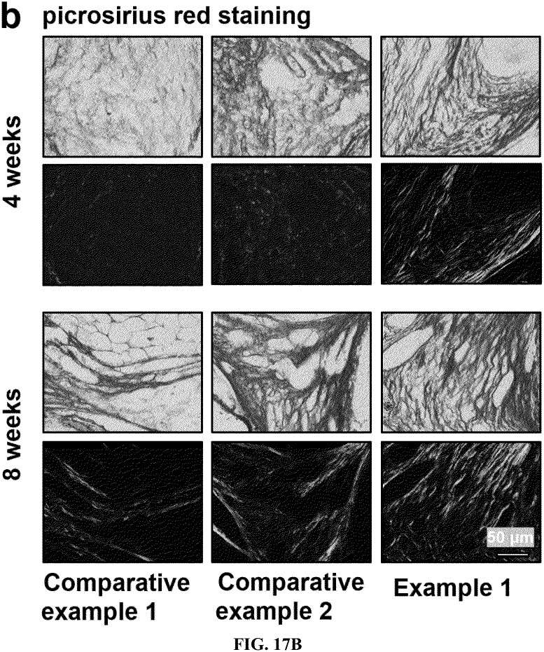

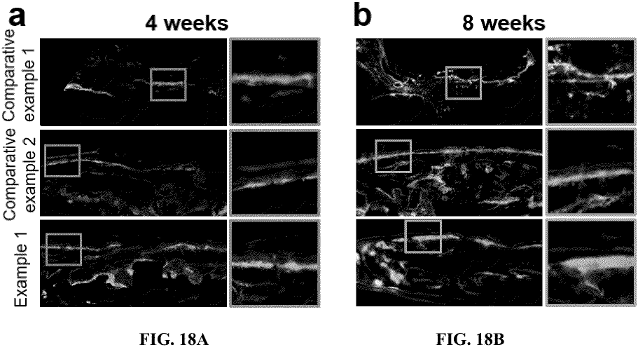

The longitudinal sections of the parietal bone defects of mice were obtained after 4 and 8 weeks of culture. The type I collagen in the longitudinal sections was detected by immunohistochemistry, and the type I collagen was detected by Sirius red staining under polarized light microscope. The result is shown in FIGS. 17A-17B. FIG. 17A is the result of IHC (brown and yellow), and FIG. 17B is the result under polarized light (red and yellow). It can be seen that the amount of type I collagen secreted by the bone-repair functional module provided in example 1 of the present invention is the largest, which is much higher than that of Comparative Example 1 and higher than that of comparative example 2.

### d. Evaluation of Bone Defect Repair Function - In situ Detection of Bone Formation

After labeling mice with new bone markers calcein and alizarin red for 7 and 5 days, respectively, longitudinal sections of parietal defects of mice at 4 and 8 weeks after implantation without decalcification were obtained and imaged with their respective sensitive fluorescence, as shown in FIGS. 18A-18B. Example 1 was much higher than comparative examples 1 and 2 in terms of fluorescence intensity, length of marker and pitch of red and yellow marker lines. This indicates that the rate of bone formation in Example 1 is accelerated.

### e. Evaluation of Bone Defect Repair Function - Osteoclast Staining

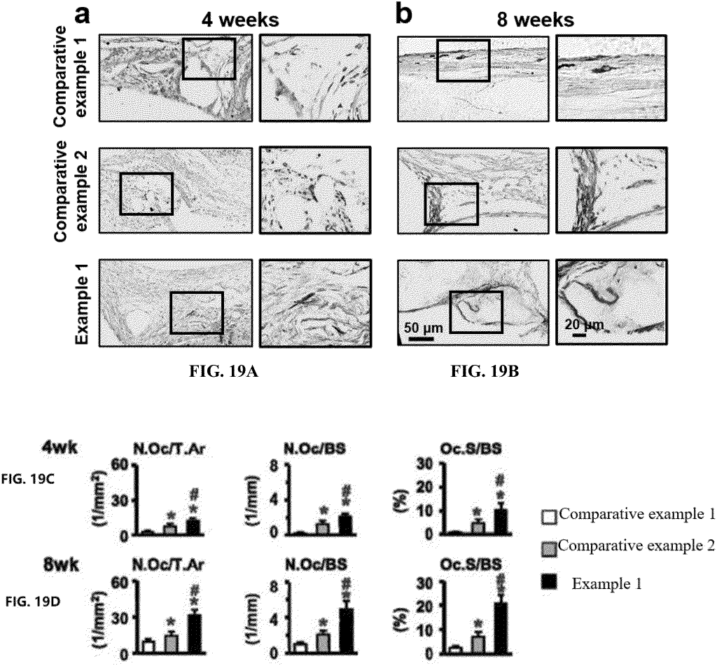

TRAP staining stained the osteoclasts in bone tissue sections as purplish red, and the background staining was green or cyan. Statistical analysis was performed according to the staining results, as shown in FIGS. 19A-19D. It can be seen that a certain amount of osteoclasts were formed in the fourth week after Wnt activation of the bone cells of example 1, which was significantly more than that of comparative example 2, while there were almost no osteoclasts in Comparative Example 1 at this time; at the 8th week, a large number of osteoclasts were formed, with a density similar to that of osteoclasts on the surface of normal bone, 1.4 times higher than that of comparative example 2 and 3.9 times higher than that of Comparative Example 1. At this time, only a few osteoclasts were found in the bone of Comparative Example 1, and their density was lower than the normal bone surface. It indicates that Wnt activation of osteocytes promoted osteoclast differentiation and made artificial bone have metabolic function. Bone with metabolic function is not only conducive to bone regeneration, but also conducive to bone health.

### f. Evaluation of Bone Defect Repair Function - Analysis of Blood Vessel Formation

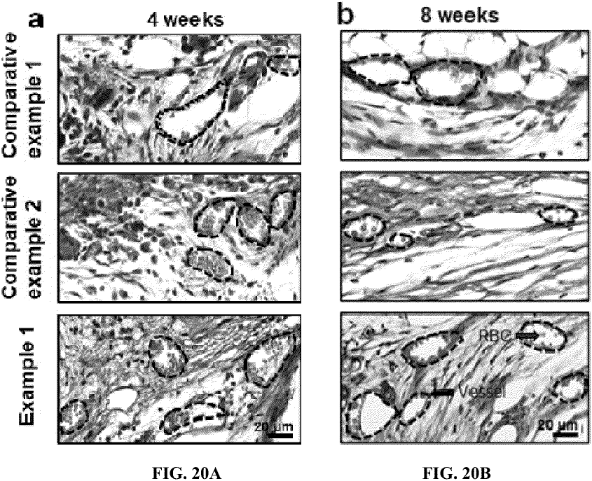

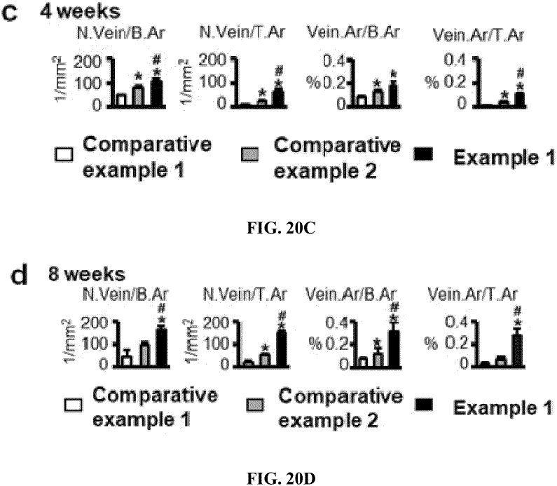

After staining the longitudinal section of the parietal bone in the mouse model, it was found that the blood vessels in Example 1 increased. Then the images were obtained by bone morphology analysis microscope, and the number and area of blood vessels were analyzed according to the results of the images, as shown in FIGS. 20A-20D. It can be seen that the number and area of blood vessels in example 1 are much higher than those in comparative examples 1 and 2.

### g. Evaluation of Bone Defect Repair Function - Analysis of Peripheral Nerve Formation

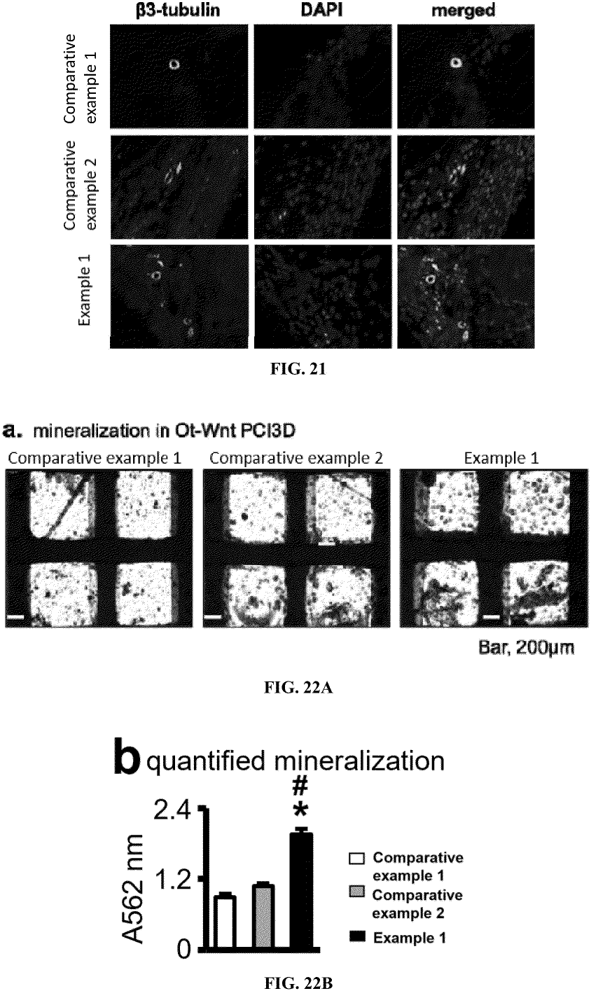

After immunostaining of the longitudinal parietal sections of the mouse model with the peripheral nerve marker β3-tubulin, it was found that the peripheral nerve in Example 1 increased, as shown in FIG. 21.

### 7.Detection of Calcium Nodules in Bone Formation

The section samples were stained with 0.1% alizarin red-Tris-HCl dye solution at 4 weeks after 10% neutral formalin fixation. Alizarin red reacted with calcium to generate dark red compounds, and the mineralization of the samples was detected by frozen sections to characterize the formation of bone nodules. For detailed experimental procedures, see the literature “Tu et al (2007) Noncanonical Wnt signaling through G protein-linked PKCδactivation promotes bone formation. Dev Cell 12(1): 113-27” and “Venugopal et al (2011) Osteoblast mineralization with composite nanofibrous substrate for bone tissue regeneration. Cell Biol Int 35(1): 73 - 80ʺ.

The above tests were carried out on example 1 and comparative examples 1 and 2, and the results are shown in FIG. 21. It can be seen that the bone-repair functional module of example 1 of the present invention has formed bone nodules after 7 days of culture, which is significantly more than that of comparative examples 1 and 2.

The above tests were carried out on example 4 and comparative examples 4 and 6, and the results of bone nodule formation experiment in the functional module of osteocyte D114 also showed that the degree of mineralization was higher than those in the two control groups (as shown in FIGS. 22A-22B).

### 8.Osteogenic Function and Mechanism of Osteoblasts Overexpressing D114 in Osteoblasts

### a. Osteogenic Function of Osteocytes Overexpressing D114 Type Bone-Repair Functional Tissue Modules

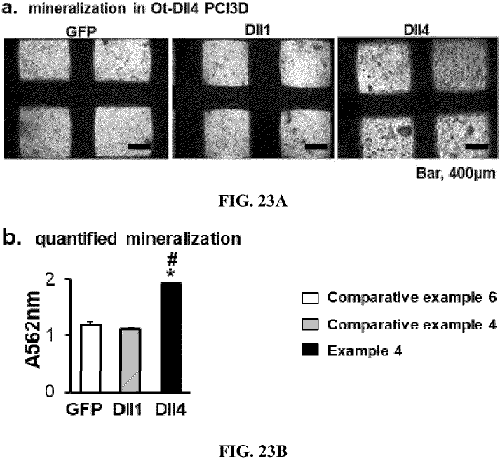

Overexpression of Notch ligand D114 by osteocytes MLO-Y4 significantly promoted osteogenic differentiation of bone marrow stromal cells ST2 when co-cultured with MLO-Y4. Both alkaline phosphatase staining (FIG. 5), biochemical activity assays (FIG. 7), and osteoblast gene expression (FIG. 9) were significantly increased and promoted bone nodule formation (FIGS. 23A-23B). It indicated that the degree of mineralization of functional modules of bone-repair in Example 4 was increased.

### b. Determination of the Relationship Between Notch Signaling Activated By D114 Osteocytes and Osteogenic Function of Osteoid Hard Tissue Modules

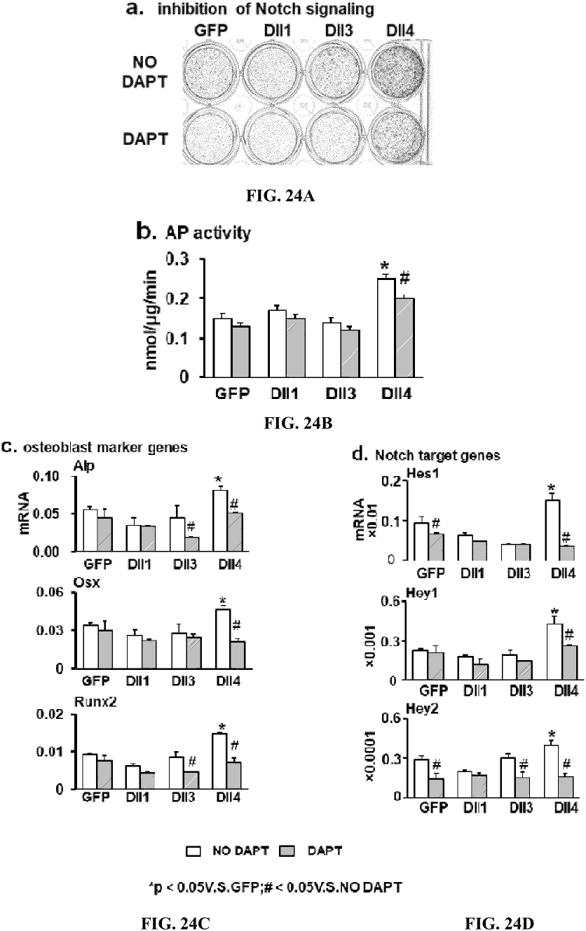

When the Notch signaling inhibitor DAPT was added to example 4 and comparative examples 4 to 6 to inhibit the Notch signaling of the bone-repair functional module, the inhibition of osteogenic differentiation and the reduction of Notch signaling by DAPT were measured. The detection results are shown in FIGS. 24A-24D. Wherein FIG. 24A and FIG. 24B are the qualitative and quantitative determination results of alkaline phosphatase activity, FIG. 24C is the expression changes of osteoblast marker genes, FIG. 24D is the expression changes of Notch signaling pathway target genes. As can be seen from the figure, DAPT significantly inhibited the background alkaline phosphatase activity of cells in the absence of DAPT at a ratio of 4-6, partially inhibited the alkaline phosphatase activity enhanced by osteocyte D114 (FIGS. 24A-24D), and completely inhibited the expression of osteoblast marker genes enhanced by osteocyte D114 (FIG. 24C). Notch signaling was completely inhibited. It indicates that D114 promotes osteogenic differentiation through Notch signaling.

### c. Osteocyte D114 Promotes Osteogenic Differentiation Through Classical Notch Signaling Mediated by Target Cell RBPjκ

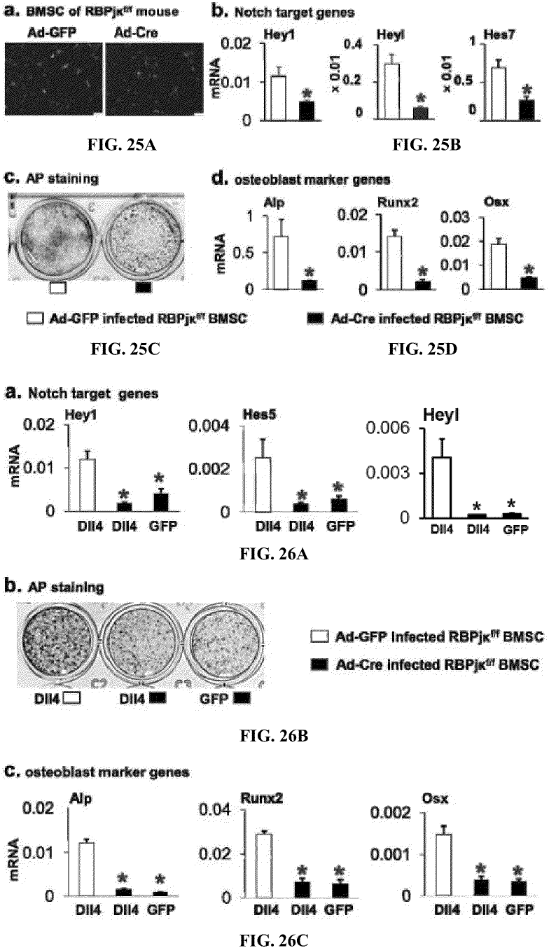

Primary BMSC from C57BL/6 strain of RBPjκ^{f/f} transgenic mice were extracted and transfected into primary BMSC cells using two recombinant adenovirus Ad-Cre and Ad-GFP(control) constructed by Ad-Easy system. Knockdown of RBPjκ gene was achieved in vitro to terminate Notch signaling. The transfected Ad-GFP was the control group of Ad-Cre. The detection results are shown in FIGS. 25A-25D, in which FIG. 25A is the detection result of fluorescence microscopy, which proves that the two recombinant adenovirus strains were successfully transfected into BMSC cells, and the transfection degree was similar; FIG. 25B is the expression of target genes of Notch signaling pathway. It can be seen that after knockdown of RBPjκ gene, the expression of target genes of Notch signaling pathway is significantly reduced; FIG. 25C is the qualitative detection result of alkaline phosphatase activity. After knockdown of RBPjκ gene, the alkaline phosphatase activity of BMSC was significantly reduced; FIG. 25D is the expression of osteoblast marker gene. It can be seen that after knockdown of RBPjκ gene, the expression level of osteoblast marker gene is significantly reduced, which proves that without RBPjκ mediated classical Notch signaling, BMSCS lose the function of osteogenic differentiation.

Then, the above transfected primary BMSCS were co-cultured with bone cell lines overexpressing Dll4, and three experimental groups were set up, namely Ad-Cre+MLO-Y4-GFP, Ad-Cre+ MLO-Y4-Dll4 and Ad-GFP+ MLO-Y4-Dll4, to judge the osteogenic effect and activation of Notch signaling. The result is shown in FIGS. 26A-26C, in which FIG. 26A is the detection of Notch signaling target gene expression level in the three experimental groups. It can be seen that osteocyte D114 cannot increase the Notch signaling intensity of BMSC after RBPjκ knockdown; FIG. 26B is the qualitative detection results of alkaline phosphatase activity, which showed that osteocyte D114 could not improve the alkaline phosphatase activity of BMSC after RBPjκ knockdown; FIG. 26C is the comparison of the expression of osteoblast marker genes. It can be seen that osteocyte D114 cannot promote the osteogenic differentiation of BMSC after RBPjκ knockdown.

From the above experimental results, it can be seen that both DAPT drug and RBPjκ knockdown greatly reduced Notch signaling and also reduced osteogenic differentiation to a very low level. It is demonstrated that osteocyte Dll4, as an independent osteogenic microenvironmental factor, promotes osteogenesis by activating classical Notch signaling mediated by Notch transcription factor RBPjκ in target cells.

### Overexpression of Dll4 in Osteocytes Promotes Angiogenesis Analysis

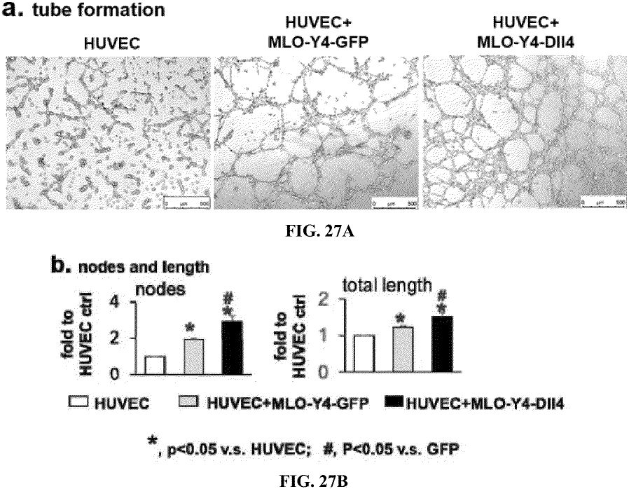

Human umbilical vein endothelial cells (HUVECs) were mixed with the transfected bone cells provided in example 4 and comparative example 6, and then the angiogenic ability was investigated. For specific experimental procedures, refer to the literature “Zhang Q et al (2019) ACE2 inhibits breast cancer angiogenesis via suppressing the VEGFa/VEGFR2/ERK pathway. JExp Clin Cancer Res 38 : 173 ” . The experimental results are shown in FIGS. 27A-27B, in which FIG. 27A is HUVEC angiogenesis test and FIG. 27B is analysis of angiogenesis mode. The results showed that only HUVEC cells without bone cells had poor angiogenic ability. Wild-type osteocytes have some ability of vasculogenesis, but not as strong as osteocytes overexpressing Dll4.

Finally, it should be noted that the above embodiments are intended only to describe the technical solution of the invention and not to restrict it; notwithstanding the detailed description of the present invention with reference to the foregoing embodiments, ordinary technicians in the field shall understand that: It may still modify the technical solution recorded in each of the foregoing embodiments, or replace some or all of the technical features thereof equally; these modifications or substitutions do not remove the nature of the corresponding technical solution from the scope of the technical solution of each embodiment of the invention.

What is claimed is:

1. A integrated 3D printing method for integrating a hard material and a cell, comprising a method for preparing a bone-repair functional module by integrating a high-strength biomedical material and the cell with a synchronous 3D printing; wherein
the high-strength biomedical material refers to the hard material with a compression strength of 2 MPa and above;
the high-strength biomedical material is printed in a form of a hard material bundle, and the cell is printed in a form of a cell bundle;
the integrated 3D printing method comprises a use of a multi-nozzle alternately printing the hard material bundle and the cell bundle, so that the hard material bundle and the cell bundle are arranged in parallel into layers, and then printed layer by layer into a three-dimensional structure with channels, printing directions of the hard material bundle and the cell bundle are perpendicular or at an angle to each other, and then the bone-repair functional module is obtained;
the multi-nozzle comprises at least two nozzles, namely a material printing nozzle and a cell printing nozzle;
the cell comprises a cell creating an osteogenic microenvironment.

2. The integrated 3D printing method according to claim 1, wherein the cell creating the osteogenic microenvironment comprises a cell related to a bone tissue formation;
wherein the cell related to the the bone tissue formation is at least one selected from the group consisting of a bone marrow stromal cell, a bone progenitor cell, a preosteoblast, an osteoblast, a bone lining cell, an osteocyte, and an osteoclast;
wherein the osteocyte is activated by a Wnt signaling and configured to be used to create the osteogenic microenvironment, promote a proliferation, an osteogenic differentiation, and a mineralization of the bone marrow stromal cell, promote a differentiation of the osteoclast, and promote a regeneration and repair of bone;
wherein a Wnt signaling activation method comprises an activation of classical Wnt/β-catenin signaling by one or more components of biomedical materials, small molecule drugs, proteins, and peptides;
wherein a number ratio of the Wnt signaling activated bone cells to the bone marrow stromal cell is 1: (2 to 8);
wherein the osteocyte overexpresses at least one osteogenic biological microenvironment factor;
wherein the at least one osteogenic biological microenvironment factor comprises a D114.

3. The integrated 3D printing method according to claim 1, wherein a printing method of the hard material bundle comprises fusing the high-strength biomedical material successively and extruding by a screw to obtain the hard material bundle; and/or a printing method of the cell bundle comprises extruding a hydrogel or a bioink wrapping the cell by a pneumatic drive to obtain the cell bundle;
wherein a melting temperature of the hard material is 30 - 200° C., a printing temperature of the hard material bundle is 30 - 200° C.;
wherein a printing temperature of the cell bundle is 4 to 37° C.;
wherein a printing speed of the 3D printing is 2 - 10 mm/s.

4. The bone-repair functional module obtained by the integrated 3D printing method according to claim 1, comprising a material unit for mechanical scaffolds, a cell unit for an osteogenic function and a pore channel; wherein a volume ratio of the material unit and the cell unit in an osteoid hard tissue module is 1:0.5 - 2, and a porosity of the osteoid hard tissue module is 20% - 80%;
wherein the pore channel comprises one or more combinations of multiple holes, buried holes, and blind holes.
wherein a printing method of the pore channel comprises a separation of adjacent hard material bundles and cell bundles, or a printing of a pore forming material, and after the printing is completed, the pore forming material is removed to form multiple pores and /or channels;
wherein the channel forming material comprises a sacrificial material;
wherein the sacrificial material comprises Pluronic F127.

5. The bone-repair functional module according to claim 4, wherein the material unit comprises a composite polymer containing hydroxyapatite;
wherein a particle size of the composite polymer containing hydroxyapatite is nanometer-scale;
wherein a polymer material used in the composite polymer containing hydroxyapatite comprises one or more combinations of polycaprolactone and its derived copolymers;
wherein a mass ratio of the composite polymer containing hydroxyapatite to the polymer material is 1:(4 - 9).

6. The bone-repair functional module according to claim 4, wherein the cell unit comprises a hydrogel or a bioink encapsulating the cell, a density of the cell in the bioink or the hydrogel ranges from 1×10⁵ to 1×10⁷ cells/ml;
wherein the bioink or the hydrogel comprises a solidifying molecule;
wherein the solidifying molecule comprises a methylacrylylated gelatin.

7. A bone defect repair material, comprises the bone-repair functional module according to claim 4 and a bone organoid, wherein the bone defect repair material is obtained after an in vitro culture;
wherein the in vitro culture comprises culturing the bone-repair functional module for 7 to 30 days in a cell medium in an incubator or a bioreactor with a volume ratio of 5% carbon dioxide and a temperature of 37° C.;
wherein the cell medium is a cell growth medium, and the bone defect repair material obtained after the in vitro culture is a functional module of bone-repair;
wherein the bone-repair functional module is cultured with an osteogenic differentiation medium, and the bone defect repair material obtained is a mineralized bone organoid;
wherein the osteogenic differentiation medium comprises dexamethasone, vitamin C, and sodium β glycerophosphate.

8. The bone defect repair material according to claim 7, wherein in the bone-repair functional module, an osteocyte overexpresses one or more osteogenic biological microenvironmental factors.

9. The bone defect repair material according to claim 8, wherein a D114 acts as a Notch signaling ligand to activate a classical Notch signaling pathway of target cells, after the target cells are activated, an intracellular fragment NICD of a Notch receptor, a Notch signaling transmitter, is generated, the intracellular fragment NICD enters a nucleus to activate a Notch signaling transcription factor RBPjκ, initiate a transcription and an expression of a Notch signaling Hes/Hey family and other target genes, thereby promoting a proliferation, a differentiation and a rapid bone formation of the target cells, and promoting endothelial cells to form blood vessels;
wherein the target cells comprise at least one of bone progenitor cells, preosteoblasts, osteoblasts, bone lining cells, bone marrow stromal cells, and/or osteoclast and its precursors;
wherein a classical Notch signaling activation method comprises activating a classical Notch signaling by one or more components of biomedical materials, small molecule drugs, proteins, and peptides.

10. A method of application of the bone-repair functional module according to claim 4 in a preparation of a tissue replacement and/or repair material;
wherein when the tissue replacement and/or repair material is cultured in vitro, cells on a surface of the tissue replacement and/or repair material have a high survival rate and a proliferation activity, and successfully achieve an osteogenic differentiation and a mineralization; after an implantation in animals, the tissue replacement and/or repair material has dual metabolic functions of bone formation and bone resorption, pro-vascular, and neurogenic functions;
a tissue comprises a hard tissue structure or a skeletal structure in a soft tissue.

11. The integrated 3D printing method according to claim 2, wherein the cell related to the the bone tissue formation is at least one selected from the group consisting of the bone marrow stromal cell and the osteocyte.

12. The integrated 3D printing method according to claim 2, wherein the number ratio of the Wnt signaling activated the bone cells to the bone marrow stromal cell is 1:4.

13. The bone-repair functional module according to claim 5, wherein the mass ratio of the composite polymer containing hydroxyapatite to the polymer material is 1:9.

14. The bone-repair functional module according to claim 6, wherein the density of the cell in the bioink or the hydrogel is 1×10⁶ cells/ml.

15. A method of application of the bone defect repair material according to claim 7 in a preparation of a tissue replacement and/or repair material;
wherein when the tissue replacement and/or repair material is cultured in vitro, cells on a surface of the tissue replacement and/or repair material have a high survival rate and a proliferation activity, and successfully achieve an osteogenic differentiation and a mineralization; after an implantation in animals, the tissue replacement and/or repair material has dual metabolic functions of bone formation and bone resorption, pro-vascular, and neurogenic functions;
a tissue comprises a hard tissue structure or a skeletal structure in a soft tissue.

16. The bone-repair functional module according to claim 4, wherein in a process of preparing the bone-repair functional module, the cell creating the osteogenic microenvironment comprises a cell related to a bone tissue formation;
wherein the cell related to the the bone tissue formation is at least one selected from the group consisting of a bone marrow stromal cell, a bone progenitor cell, a preosteoblast, an osteoblast, a bone lining cell, an osteocyte, and an osteoclast;
wherein the osteocyte is activated by a Wnt signaling and configured to be used to create the osteogenic microenvironment, promote a proliferation, an osteogenic differentiation, and a mineralization of the bone marrow stromal cell, promote a differentiation of the osteoclast, and promote a regeneration and repair of bone;
wherein a Wnt signaling activation method comprises an activation of classical Wnt/β-catenin signaling by one or more components of biomedical materials, small molecule drugs, proteins, and peptides;
wherein a number ratio of the Wnt signaling activated bone cells to the bone marrow stromal cell is 1: (2 to 8);
wherein the osteocyte overexpresses at least one osteogenic biological microenvironment factor;
wherein the at least one osteogenic biological microenvironment factor comprises a D114.

17. The bone-repair functional module according to claim 4, wherein in a process of preparing the bone-repair functional module, a printing method of the hard material bundle comprises fusing the high-strength biomedical material successively and extruding by a screw to obtain the hard material bundle; and/or a printing method of the cell bundle comprises extruding a hydrogel or a bioink wrapping the cell by a pneumatic drive to obtain the cell bundle;
wherein a melting temperature of the hard material is 30 - 200° C., a printing temperature of the hard material bundle is 30 - 200° C.;
wherein a printing temperature of the cell bundle is 4 to 37° C.;
wherein a printing speed of the 3D printing is 2 - 10 mm/s.
